(19) United States
(12) Patent Application Publication
(10) Pub. No.: US 2026/0013736 A1
(43) Pub. Date: Jan. 15, 2026

(54) APPARATUS FOR BLOOD PRESSURE MONITORING, AND A METHOD FOR BLOOD PRESSURE MONITORING USING THE SAME

(71) Applicant: UIF (University Industry Foundation), Yonsei University, Seoul (KR)

(72) Inventors: Shin Ill KANG, Seoul (KR); Joon Sang LEE, Seoul (KR); Min Soo PARK, Seoul (KR); Jeong Eun SHIN, Seoul (KR); Jung Ho HAN, Seoul (KR); Dong Hyun KIM, Seoul (KR); Eun Joo CHAE, Seoul (KR)

(21) Appl. No.: 19/037,221

(22) Filed: Jan. 26, 2025

(30) Foreign Application Priority Data
Jul. 10, 2024 (KR) 10-2024-0090853

(51) Int. Cl. A61B 5/021; A61B 5/00; A61B 5/11

(52) U.S. Cl. CPC A61B 5/021; A61B 5/0082; A61B 5/1102

(57) ABSTRACT

An apparatus for blood pressure monitoring comprises: a blood vessel deformation detection sensor unit for monitoring blood volume changes and blood flow in blood vessels of a part of the user's body; a ballistocardiogram sensor unit installed adjacent to the user's back to measure the user's heartbeat; a blood vessel analysis unit for receiving signals from the blood vessel deformation detection sensor unit, analyzing them, and generating a graph; a ballistocardiogram analysis unit for receiving signals from the ballistocardiogram sensor unit, analyzing them, and generating a graph; a calculation unit for receiving data from the blood vessel analysis unit and the ballistocardiogram analysis unit and deriving at least one parameter; and a machine learning unit for receiving data on the parameter from the calculation unit and performing machine learning to derive blood pressure values.

### CROSS-REFERENCE TO RELATED APPLICATIONS

This application claims priority under 35 U.S.C. § 119 (a) to Korean Patent Application No. 10-2024-0090853, filed on Jul. 10, 2024, with the Korean Intellectual Property Office, the disclosure of which is incorporated herein in its entirety by reference.

### BACKGROUND

### 1. Technical Field

The present disclosure relates to an apparatus and method for blood pressure monitoring, and more particularly, to a technology, which simply measures blood pressure without contact and simultaneously improves the accuracy of blood pressure monitoring by using the configuration of the sensor, etc.

### 2. Description of the Related Art

Blood pressure is caused by heartbeats due to the pressure of blood facing the walls of arterial blood vessels. Blood pressure monitoring is an important vital sign that reflects the state of cardiovascular system function, which is important for maintaining human life, and provides objective data to determine changes in health. Therefore, research on monitoring methods that do not cause physical burden to patients has been continuously conducted.

An example of this is blood pressure monitoring technology that uses bio-signals obtained by attaching sensors directly to the body, and methods such as monitoring blood pressure by calculating blood flow velocity in blood vessels through bio-signal data including electrocardiogram signals are being used.

Conventional technologies can monitor blood pressure by contacting the sensor with the body, but they have limitations such as changes in the sleeping environment caused by contact and the occurrence of abnormalities when attaching or detaching the sensor.

In addition, existing representative methods for examining blood pressure include non-invasive methods such as adjusting the pressure of the sphygmomanometer cuff to measure the pressure in the area where Kortkoff sounds are heard, and invasive methods such as inserting a catheter into a blood vessel to measure pressure.

However, there are limitations in that, when using a sphygmomanometer cuff, continuous measurement is not possible, errors may occur depending on the size of the cuff and the skill of the measurer, and when measuring blood pressure through insertion of a catheter into a blood vessel, the cost is high and complications may occur.

In Korean Patent Publication No. 10-2020-0078795 (Title: APPARATUS AND METHOD FOR ESTIMATING BLOOD PRESSURE), an apparatus is disclosed that includes a BCG (ballistocardiogram) sensor configured to measure a BCG signal from a user, and a processor configured to obtain blood pressure-related features from the measured BCG signal, according to sensing characteristics of the BCG sensor, and estimate a blood pressure of the user based on the obtained blood pressure-related features.

### SUMMARY OF THE INVENTION

An object of the present disclosure to solve the above problems is to provide a technology, which simply measures blood pressure without contact and simultaneously improves the accuracy of blood pressure monitoring by using the configuration of the sensor, etc.

The technical objects to be achieved by the present disclosure are not limited to as described-above, and other technical objects which are not described will be clearly understood by a person who has ordinary knowledge in a technical field to which the present disclosure pertains from the following description.

A configuration of the present disclosure for achieving the above objects includes: a blood vessel deformation detection sensor unit for monitoring blood volume changes and blood flow in blood vessels of a part of the user's body; a ballistocardiogram sensor unit installed adjacent to the user's back to measure the user's heartbeat; a blood vessel analysis unit for receiving signals from the blood vessel deformation detection sensor unit, analyzing them, and generating a graph; a ballistocardiogram analysis unit for receiving signals from the ballistocardiogram sensor unit, analyzing them, and generating a graph; a calculation unit for receiving data from the blood vessel analysis unit and the ballistocardiogram analysis unit and deriving at least one parameter; and a machine learning unit for receiving data on the parameter from the calculation unit and performing machine learning to derive blood pressure values.

In an embodiment of the present disclosure, the blood vessel deformation detection sensor unit may be equipped with an imaging sensor for imaging blood vessels on a part of the user's body.

In an embodiment of the present disclosure, the ballistocardiogram sensor unit may comprise at least one ballistocardiogram sensor, and the ballistocardiogram sensor may be an accelerometer, a force sensor, a pressure sensor, or an optical fiber sensor.

In an embodiment of the present disclosure, a plurality of ballistocardiogram sensors may be arranged to form rows and columns.

In an embodiment of the present disclosure, the calculation unit may derive cardiac output and pulse transit time (PTT) as parameters using data received from the blood vessel analysis unit and the ballistocardiogram analysis unit.

In an embodiment of the present disclosure, the calculation unit may store the user's body information as a parameter.

In an embodiment of the present disclosure, the calculation unit may derive the pulse transit time (PTT) using graph data of the blood vessel analysis unit and graph data of the ballistocardiogram analysis unit.

In an embodiment of the present disclosure, the blood vessel analysis unit may generate a graph of changes in blood volume of blood vessels on a part of the user's body.

In an embodiment of the present disclosure, the ballistocardiogram analysis unit may generate a graph of the user's heartbeat.

A configuration of the present disclosure for achieving the above objects includes: a preparation step in which a user lies down on an upper surface of a bed; a blood vessel measurement step in which the blood vessel deformation detection sensor unit performs imaging of blood vessels of a part of the user's body to measure blood volume changes; a ballistocardiogram measurement step in which the ballistocardiogram sensor unit measures the user's heartbeat; an analysis step in which each of the blood vessel analysis unit and the ballistocardiogram analysis unit generates a graph; a calculation step in which the calculation unit receives data from the blood vessel analysis unit and the ballistocardiogram analysis unit and derives at least one parameter; and a machine learning step in which the calculation unit receives data on the parameter and performs machine learning to derive the user's blood pressure value.

The effect of the present disclosure according to the above configuration is that, by collecting data from the user's body in a non-contact manner and using the data to derive the user's blood pressure value, the burden of blood pressure measurement on the user's body is reduced, thereby enabling convenient and continuous measurement.

In addition, the effect of the present disclosure is that precise blood pressure measurement can be implemented by performing calculations using changes in blood volume of blood vessels and multiple ballistocardiogram information, and deriving the user's blood pressure value through machine learning using the calculation data.

The effects of the embodiments of the present disclosure are not limited to the above-mentioned effects, and it should be understood that the effects of the present disclosure include all effects that could be inferred from the configuration of the invention described in the detailed description of the invention or the appended claims.

### BRIEF DESCRIPTION OF THE DRAWINGS

FIG. 1 is a block diagram of a configuration of a monitoring apparatus according to an embodiment of the present disclosure.

FIG. 2 is an image obtained by a blood vessel deformation detection sensor unit according to an embodiment of the present disclosure.

FIG. 3 is an image of a process of performing PTT derivation in a calculation unit according to an embodiment of the present disclosure.

FIG. 4 is an image of a measurement location of a blood vessel deformation detection sensor unit and a ballistocardiogram sensor unit according to an embodiment of the present disclosure.

### DETAILED DESCRIPTION OF THE INVENTION

Hereinafter, embodiments of the present disclosure will be explained with reference to the accompanying drawings. The invention, however, may be implemented in various different ways or forms, and should not be construed as limited to the embodiments set forth herein. Also, in order to clearly explain the embodiments of the present disclosure, parts that are not related to the explanation are omitted in the drawings, and like reference numerals are used to refer to like elements throughout.

Throughout the specification, when a certain part is referred to as being “connected” or “coupled” to another part, it includes not only “directly connected” but also “indirectly connected” with another member therebetween. Further, when a certain part “includes” a certain component, unless described to the contrary, this means that other components may not be excluded, but other components may be further provided.

The terminology used herein is for the purpose of describing various embodiments only and is not intended to be limiting. As used herein, the singular forms are intended to include the plural forms as well, unless the context clearly indicates otherwise. It will be further understood that the terms “comprises” and/or “has,” when used in this specification, specify the presence of a stated feature, number, step, operation, component, element, or combination thereof, but do not preclude the presence or addition of one or more other features, numbers, steps, operations, components, elements, or combinations thereof.

Hereinafter, the present disclosure will be described in detail with reference to the accompanying drawings.

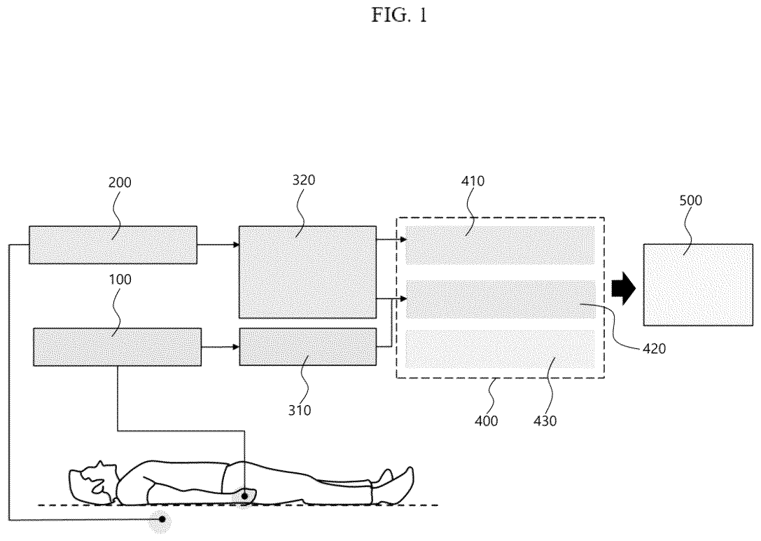

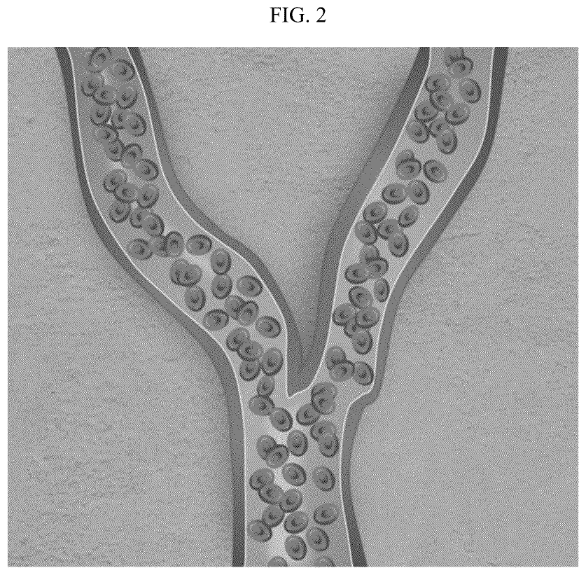

FIG. 1 is a block diagram of a configuration of a monitoring apparatus according to an embodiment of the present disclosure, and FIG. 2 is an image obtained by a blood vessel deformation detection sensor unit 100 according to an embodiment of the present disclosure. In FIG. 1, the measurement location of the blood vessel deformation detection sensor unit 100 and the measurement location of the ballistocardiogram sensor unit 200 are indicated for convenience of understanding.

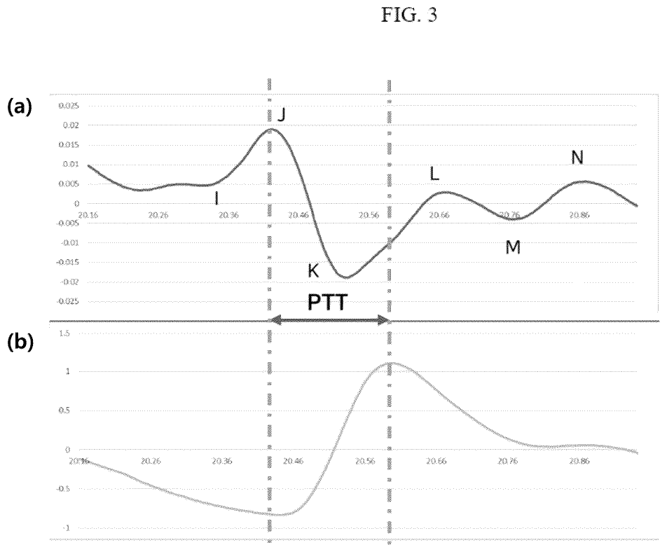

In addition, FIG. 3 is an image of a process of performing PTT derivation in a calculation unit 400 according to an embodiment of the present disclosure. Here, (a) of FIG. 3 is for a graph generated by a ballistocardiogram analysis unit 320 using a signal from a ballistocardiogram sensor unit 200, and (b) of FIG. 3 is for a graph generated by a blood vessel analysis unit 310 using a signal from a blood vessel deformation detection sensor unit 100.

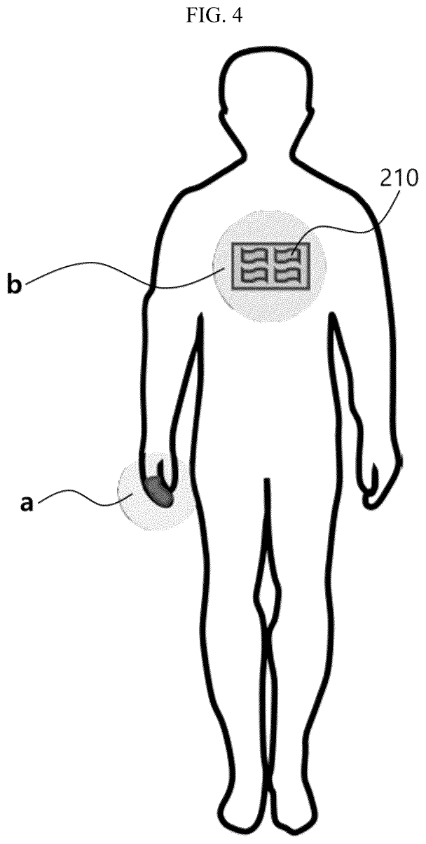

In addition, FIG. 4 is an image of a measurement location of a blood vessel deformation detection sensor unit 100 and a ballistocardiogram sensor unit 200 according to an embodiment of the present disclosure. In FIG. 4, area a is the measurement location of the blood vessel deformation detection sensor unit 100 and area b is the measurement location of the ballistocardiogram sensor unit 200.

As shown in FIGS. 1 to 4, the monitoring apparatus of the present disclosure includes: a blood vessel deformation detection sensor unit 100 for monitoring blood volume changes and blood flow in blood vessels of a part of the user's body; a ballistocardiogram sensor unit 200 installed adjacent to the user's back to measure the user's heartbeat; a blood vessel analysis unit 310 for receiving signals from the blood vessel deformation detection sensor unit 100, analyzing them, and generating a graph; a ballistocardiogram analysis unit 320 for receiving signals from the ballistocardiogram sensor unit 200, analyzing them, and generating a graph; a calculation unit 400 for receiving data from the blood vessel analysis unit 310 and the ballistocardiogram analysis unit 320 and deriving at least one parameter; and a machine learning unit 500 for receiving data on the parameter from the calculation unit 400 and performing machine learning to derive blood pressure values.

The blood vessel deformation detection sensor unit 100 may be equipped with an imaging sensor for imaging blood vessels in a part of the user's body.

An imaging sensor used when constructing an imaging system using phase contrast microscopy, differential interference contrast microscopy, oblique back illumination microscopy, etc. can be used as the imaging sensor mentioned above, and specifically, a CCD sensor, a CMOS sensor or the like can be used.

The blood vessel deformation detection sensor unit 100 may be formed by combining an imaging sensor and a microscope, and may monitor and track changes in blood vessel volume in a sensing area, which is a part of the user's body, through real-time image acquisition by the imaging sensor.

In addition, such a blood vessel deformation detection sensor unit 100 may monitor blood flow, and by using this, images related to changes in blood flow amount and flow distance for a certain period of time can be obtained.

As another embodiment, the blood vessel deformation detection sensor unit 100 may use a PPG (Photoplethysmography) sensor to measure blood volume changes in blood vessels of a part of the user's body. When using a PPG sensor, the change in blood volume due to contraction and relaxation of the heart can be measured using an optical sensor.

In the above, the part of the user's body may be adjacent to a finger, the tip of a finger, the tip of a toe, an ear, etc., and generally, measurement of blood vessels can be performed at the tip of the user's finger using the blood vessel deformation detection sensor unit 100 in a non-contact manner. However, the part of the body is not limited thereto.

The ballistocardiogram sensor unit 200 may be equipped with at least one ballistocardiogram sensor 210 for measuring ballistocardiogram (BCG), and the ballistocardiogram sensor 210 may be an accelerometer, a force sensor, a pressure sensor, or an optical fiber sensor. Accordingly, the ballistocardiogram sensor 210 can detect vibrations generated in the user's back area due to heartbeat when placed adjacent to the user's back and generate a signal using this.

The monitoring apparatus of the present disclosure may include a bed that provides a space where a user can lie down, and the ballistocardiogram sensor 210 may be placed on a portion of the upper surface of the bed where the user lies down, which is in contact with the user's back. Here, the ballistocardiogram sensor 210 may be installed at a location adjacent to the upper surface of the bed within the bed.

In this case, a plurality of ballistocardiogram sensors 210 may be arranged to form rows and columns. As shown in FIG. 4, the direction of the user's shoulder width may be the row direction, and the direction of the user's height may be the column direction, and by arranging a plurality of ballistocardiogram sensors 210 in this manner, a plurality of signals by each signal of the plurality of ballistocardiogram sensors 210 may be transmitted to the ballistocardiogram analysis unit 320.

The blood vessel analysis unit 310 may generate a graph of blood volume changes in blood vessels of a part of the user's body. In addition, the ballistocardiogram analysis unit 320 may generate a graph of the user's heartbeat.

As described above, the blood vessel deformation detection sensor unit 100 may continuously transmit images of blood volume changes and blood flow to the blood volume analysis unit, and the blood volume analysis unit may perform image analysis to generate a graph of blood volume changes.

Specifically, the blood vessel analysis unit 310 may store a program for an image analysis model using machine learning, and the blood vessel analysis unit 310 may set the maximum value of blood volume change to 1 through image analysis using the image analysis model, calculate the minimum value and the change value between the maximum and minimum values based on the maximum value, and use this to quantify the blood volume change over time to generate a blood volume change graph such as (b) of FIG. 3.

Here, models using regression analysis, artificial neural networks, etc. may be used as image analysis models, and a detailed description of the technology for measuring changes in targets through image analysis in this way will be omitted as it is a prior art.

As described above, a plurality of ballistocardiogram signals, which are a plurality of signals by a plurality of ballistocardiogram sensors 210, may be transmitted to the ballistocardiogram analysis unit 320, and the ballistocardiogram analysis unit 320 may perform analysis on each of the plurality of ballistocardiogram signals to select a stable signal.

As a specific embodiment, the ballistocardiogram analysis unit 320 may derive the range of variation between the maximum and minimum values in each cycle of a ballistocardiogram signal and calculate the average value of the range of variation in a plurality of cycles.

Alternatively, as another embodiment, a stable signal can be selected using signal quality assessment, outlier detection, etc.

However, the method for selecting a stable signal is not limited to the method of the above-described embodiment, and other methods can of course be used.

The ballistocardiogram analysis unit 320 may generate a graph such as that in (a) of FIG. 3 by using a stable signal selected as described above among a plurality of ballistocardiogram signals to generate a graph. In addition, the ballistocardiogram analysis unit 320 may display J waves, etc. in the selected graph, which is a heart rate graph.

The calculation unit 400 may derive cardiac output and pulse transit time (PTT) as parameters using data received from the blood vessel analysis unit 310 and the ballistocardiogram analysis unit 320. In addition, the calculation unit 400 may derive pulse transit time (PTT) using graph data from the blood vessel analysis unit 310 and graph data from the ballistocardiogram analysis unit 320.

Additionally, the calculation unit 400 may store the user's body information as a parameter.

To this end, the calculation unit 400 may include a cardiac output calculator 410 that calculates cardiac output, a PTT calculator 420 that calculates pulse transit time, and a body calculator 430 that stores the user's body information and performs calculations using the same. Here, the user's body information may include information about the user's age, weight, height, gender, etc.

In the cardiac output calculator 410, the heart rate graph information for the heart rate as described above may be received from the ballistocardiogram analysis unit 320, along with information on the amplitude of J wave and the timing of J wave according to the graph. In addition, the cardiac output calculator 410 may calculate the cardiac output using the following [Equation 1].

Cardiac output (ml/min)=Stroke Volume (ml/beat)×Heartrate (beats/min) [Equation 1]

Here, Cardiac output can be the amount of blood the heart pushes out in one minute. Also, Heartrate is the heart rate.

In addition, Stroke Volume is the amount of blood pushed out when the ventricle contracts once, and is a value related to the size of the J wave. Data on the correlation between the size of the J wave and the Stroke Volume as described above may be stored in advance in the cardiac output calculator 410, and the cardiac output calculator 410 may select the Stroke Volume value using the data and the J wave value.

In the PTT calculator 420, a graph of heartbeat may be received from the ballistocardiogram analysis unit 320, and a graph of blood volume change may be received from the blood vessel analysis unit 310. In addition, as shown in FIG. 3, the PTT calculator 420 may calculate the time difference between the J wave occurrence time and the blood volume maximum occurrence time, and derive this time difference as a pulse transit time (PTT) value.

Alternatively, the time difference between the feature points of each graph may be derived as the pulse transit time (PTT). Specifically, the pulse transit time (PTT) may be derived using the time difference between the J wave occurrence time and the maximum blood volume change occurrence time, or the time difference between the K wave and the maximum blood volume occurrence time, or the like.

The body calculator 430 may derive the BMI (body mass index) using the user's body information as described above and the following [Equation 2].

BMI=weight (kg)/[height (m)]² [Equation 2]

Here, weight is the user's body weight and height is the user's height.

The machine learning unit 500 may receive various data, such as cardiac output data, pulse transit time data, BMI data, and user's body information, as data for the above parameters from the calculation unit 400, and may derive a blood pressure value by performing machine learning using the data received in this manner.

To this end, a learning model using an artificial neural network may be stored in the machine learning unit 500, and a feedforward neural network, a convolutional neural network (CNN), a recurrent neural network (RNN), etc. may be used as the artificial neural network.

The machine learning unit 500 may use data obtained in a different manner than the blood pressure value derived using the monitoring apparatus of the present disclosure as training data.

Here, the data obtained in a different manner may be data obtained using a conventional blood pressure measurement method, such as information obtained using an ECG electrocardiogram sensor, a belt-type respiration measurement sensor, and an automatic blood pressure measuring device.

In the machine learning unit 500, machine learning may be performed using the above-described data using a learning model, and then blood pressure may be measured using the data of the monitoring apparatus of the present disclosure obtained in a non-contact manner as described above.

When using the monitoring apparatus of the present disclosure as described above, data is collected from the user's body in a non-contact manner and the user's blood pressure value is derived using the collected data, thereby reducing the burden of blood pressure measurement on the user's body and enabling convenient and continuous measurement.

In addition, when using the monitoring apparatus of the present disclosure, calculations are performed using changes in blood volume of blood vessels and a plurality of ballistocardiogram information, and the user's blood pressure value is derived by machine learning using the calculated data, so that precise blood pressure value measurement can be implemented.

Hereinafter, a monitoring method of the present disclosure will be described. In the monitoring method of the present disclosure, a preparation step; a blood vessel measurement step; a ballistocardiogram measurement step; an analysis step; a calculation step; and a machine learning step may be performed.

In the preparation step, the user may lie down on the upper surface of the bed.

In the blood vessel measurement step, the blood vessel deformation detection sensor unit 100 may measure blood volume changes by performing imaging on blood vessels of a part of the user's body. Next, in the ballistocardiogram measurement step, the ballistocardiogram sensor unit 200 may measure the user's heart rate.

In addition, in the analysis step, graphs may be generated from each of the blood vessel analysis unit 310 and the ballistocardiogram analysis unit 320. And, in the calculation step, the calculation unit 400 may receive data from the blood vessel analysis unit 310 and the ballistocardiogram analysis unit 320 and derive at least one parameter.

Afterwards, in the machine learning step, data on parameters may be received from the calculation unit 400 and machine learning may be performed to derive the user's blood pressure value.

A description on what is not described in relation to the monitoring method of the present disclosure may be the same as the above description about the monitoring apparatus of the present disclosure.

The foregoing description of the present disclosure is intended for exemplifications, and it will be understood by those skilled in the art that the present disclosure may be easily modified in other specific forms without changing the technical idea and or essential features of the present disclosure. Therefore, it should be understood that the embodiments described above are exemplary in all aspects and not restrictive. For example, each component described as a single type may be implemented in a distributed manner, and similarly, components described as distributed may be implemented in a combined form.

The scope of the present disclosure is shown by the following claims, and all changes or modifications derived from the meaning and scope of the claims and their equivalents should be construed as being included in the scope of the present disclosure.

What is claimed is:

1. An apparatus for blood pressure monitoring, comprising:
a blood vessel deformation detection sensor unit for monitoring blood volume changes and blood flow in blood vessels of a part of the user's body;
a ballistocardiogram sensor unit installed adjacent to the user's back to measure the user's heartbeat;
a blood vessel analysis unit for receiving signals from the blood vessel deformation detection sensor unit, analyzing them, and generating a graph;
a ballistocardiogram analysis unit for receiving signals from the ballistocardiogram sensor unit, analyzing them, and generating a graph;
a calculation unit for receiving data from the blood vessel analysis unit and the ballistocardiogram analysis unit and deriving at least one parameter; and
a machine learning unit for receiving data on the parameter from the calculation unit and performing machine learning to derive blood pressure values.

2. The apparatus for blood pressure monitoring according to claim 1,
wherein the blood vessel deformation detection sensor unit is equipped with an imaging sensor for imaging blood vessels on a part of the user's body.

3. The apparatus for blood pressure monitoring according to claim 1,
wherein the ballistocardiogram sensor unit comprises at least one ballistocardiogram sensor, and the ballistocardiogram sensor is an accelerometer, a force sensor, a pressure sensor, or an optical fiber sensor.

4. The apparatus for blood pressure monitoring according to claim 1,
wherein a plurality of ballistocardiogram sensors are arranged to form rows and columns.

5. The apparatus for blood pressure monitoring according to claim 1,
wherein the calculation unit derives cardiac output and pulse transit time (PTT) as parameters using data received from the blood vessel analysis unit and the ballistocardiogram analysis unit.

6. The apparatus for blood pressure monitoring according to claim 5,
wherein the calculation unit stores the user's body information as a parameter.

7. The apparatus for blood pressure monitoring according to claim 6,
wherein the calculation unit derives the pulse transit time (PTT) using graph data of the blood vessel analysis unit and graph data of the ballistocardiogram analysis unit

8. The apparatus for blood pressure monitoring according to claim 1,
wherein the blood vessel analysis unit generates a graph of changes in blood volume of blood vessels on a part of the user's body.

9. The apparatus for blood pressure monitoring according to claim 1,
wherein the ballistocardiogram analysis unit generates a graph of the user's heartbeat.

10. A method for blood pressure monitoring using the apparatus for blood pressure monitoring according to claim 1, comprising:
a preparation step in which a user lies down on an upper surface of a bed;
a blood vessel measurement step in which the blood vessel deformation detection sensor unit performs imaging of blood vessels of a part of the user's body to measure blood volume changes;
a ballistocardiogram measurement step in which the ballistocardiogram sensor unit measures the user's heartbeat;
an analysis step in which each of the blood vessel analysis unit and the ballistocardiogram analysis unit generates a graph;
a calculation step in which the calculation unit receives data from the blood vessel analysis unit and the ballistocardiogram analysis unit and derives at least one parameter; and
a machine learning step in which the calculation unit receives data on the parameter and performs machine learning to derive the user's blood pressure value.
